(19) United States
(12) Patent Application Publication
(10) Pub. No.: US 2023/0136143 A1
(43) Pub. Date: May 4, 2023

(54) UPPER LIMB REHABILITATION TRAINING SYSTEM INTEGRATING MULTI-SOURCE STIMULATION

(71) Applicant: THE FIRST AFFILIATED HOSPITAL, ZHEJIANG UNIVERSITY SCHOOL OF MEDICINE, WUHAN (CN)

(72) Inventors: ZUOBING CHEN, HANGZHOU (CN); BINJIE QI, HANGZHOU (CN); XUEQIONG ZHU, HANGZHOU (CN); DAMING WANG, HANGZHOU (CN); XIAOHONG WU, HANGZHOU (CN)

(21) Appl. No.: 17/903,795

(22) Filed: Sep. 6, 2022

(30) Foreign Application Priority Data
Nov. 2, 2021 (CN) 2021112891064

(51) Int. Cl. A61H 1/02; A61H 7/00; A61F 7/00; A61N 1/36

(52) U.S. Cl. CPC A61H 1/0281; A61H 7/001; A61F 7/007; A61N 1/36003; A61N 1/36139; A61H 2230/085; A61H 2201/123; A61H 2201/10; A61H 2201/0207; A61F 2007/0086; A61F 2007/0096; A61H 2201/5043; A61H 2201/14

(57) ABSTRACT

An upper limb rehabilitation training system integrating multi-source stimulation is provided and includes an upper limb rehabilitation body provided with first through fourth rigid rings, the first through fourth rigid rings are fixed with first rope knots, second rope knots, third rope knots and fourth rope knots respectively. The upper limb rehabilitation training system integrating multi-source stimulation simulates working principles of muscle more authentically by using linear drive method, and complies with laws of human kinematics. The linear drive method reduces many complex mechanical structures, and makes process of force transmission very easy. The upper limb rehabilitation training system integrating multi-source stimulation reduces weight of a rehabilitative robot, improves wearing comfort, and provides a basic guarantee for patients to devote themselves to rehabilitation training.

### TECHNICAL FIELD

The disclosure relates to a field of upper limb rehabilitation training, and more particularly to an upper limb rehabilitation training system integrating multi-source stimulation.

### BACKGROUND

Abundant clinical practice has proved that a maximum recovery of limb movement can be achieved by timely rehabilitation training and fully motivating self-restoration of human tissue after an injury of limb movement. Traditional rehabilitation treatment is made by physiotherapists to assist patients to carry out rehabilitation training, and aims to rehabilitate by artificially driving limbs, which needs a large amount of manpower, and has an undesirable effect. However, a rehabilitative robot has incomparable advantages in accuracy, stability, strength and so on, which can better assist patients to complete rehabilitation training. Although the patients can perform many simple movements like a normal person with an assistance of the rehabilitative robot, coordination between muscle tissue and brain awareness still has not been significantly improved during a long-term training. Recovery period of most patients is quite long, but final effect is not very satisfactory.

It is not difficult to see from current researches that existing upper limb rehabilitation training systems adopt a single treatment plan, mostly resort to outside force to assist limb movement, preventing muscle motor function degeneration, which makes it difficult to bring into play patients' subjective initiative. Some upper limb rehabilitation training systems have introduced supporting software and provided a virtual rehabilitation training scene for patients, but still failed to achieve a satisfactory effect.

### SUMMARY

In order to solve deficiencies of the related art, the disclosure combines multiple rehabilitation treatments to achieve an upper limb rehabilitation training system integrating multiple interventions.

In order to achieve the above purposes, the disclosure provides an upper limb rehabilitation training system integrating multi-source stimulation, including an upper limb rehabilitation body. The upper limb rehabilitation body is provided with rigid rings A through D, which are provided with first rope knots, second rope knots, third rope knots and fourth rope knots respectively, and connected to ends of ropes and other ends of the ropes are fixedly connected to micro servo motors. The upper limb rehabilitation body is further provided with a closed-loop myoelectric functional rehabilitation system, a visual stimulation rehabilitation system and a nerve system.

In an illustrated embodiment, four sets of the ropes are provided between the rigid ring A and the rigid ring B, two sets of the ropes are provided between the rigid ring B and the rigid ring C, and four sets of the ropes are provided between the rigid ring C and the rigid ring D.

In an illustrated embodiment, the closed-loop myoelectric functional rehabilitation system is provided with a myoelectric signal collector, an electric heating bandage and a stimulation electrode piece. An output end of the myoelectric signal collector is connected to an input end of the stimulation electrode piece, and an output end of the stimulation electrode piece is connected to an input end of the electric heating bandage.

In an illustrated embodiment, the electric heating bandage adopts hot compress method with temperature controlled, and is provided with a temperature sensor therein.

In an illustrated embodiment, the visual stimulation rehabilitation system is provided with an external visual stimulation module, a remodeling damaged nerve module, a cerebral cortex emitting command module, and a stimulation muscle tissue module, which are all embodied by at least one processor and at least one memory coupled to the at least one processor, and the at least one memory stores programs executable by the at least one processor. An output end of the external visual stimulation module is connected to an input end of the remodeling damaged nerve module, an output end of the remodeling damaged nerve module is connected to an input end of the cerebral cortex emitting command module, an output end of the cerebral cortex emitting command module is connected to an input end of the stimulation muscle tissue module.

In an illustrated embodiment, the nerve system is provided with a nerve center module, a nerve pathway module, and a muscle tissue module, which are all embodied by at least one processor and at least one memory coupled to the at least one processor, and the at least one memory stores programs executable by the at least one processor. The nerve center module is bi-directionally connected to an input end and an output end of the nerve pathway module, the nerve pathway module is bi-directionally connected to an input end and an output end of the muscle tissue module, and the output end of the muscle tissue module is connected to the input end of the stimulation muscle tissue module.

In an illustrated embodiment, the external visual stimulation module adopts one of methods of VR (Virtual Reality) and AR (Augmented Reality). The output end of the external visual stimulation module is connected to an output end of the nerve center module.

In an illustrated embodiment, the micro servo motors are provided on a rehabilitation system motor platform. A side of the rehabilitation system motor platform is provided with pulleys. The other ends of the ropes are connected to the micro servo motors through the pulleys.

The disclosure provides the upper limb rehabilitation training system integrating multi-source stimulation. Compared with the related art, the beneficial effects of the disclosure are:

(1) the upper limb rehabilitation training system integrating multi-source stimulation bases on medical essence of limb movement rehabilitation, links a concept of nerve remodeling with rehabilitative robot researches, and combines functional electrical stimulation, thermal stimulation, audio-visual fusion stimulation, and external force-assisted movement, aiming to rehabilitate upper limb function, realizing an upper limb rehabilitation training system integrating multiple interventions, achieving real-time monitoring of a rehabilitation process, improving human-machine coordination, and effectively improving an effectiveness of rehabilitation treatment.

(2) the upper limb rehabilitation training system integrating multi-source stimulation adopts linear drive method, which simulates working principles of muscle more authentically, complies with laws of human kinematics, reduces many complex mechanical structures, and makes process of force transmission easier. The technical solutions of the disclosure not only reduces weight of the rehabilitative robot itself, but also improves comfort of wearing, and provides a basic guarantee for patients to devote themselves to rehabilitation training.

(3) the upper limb rehabilitation training system integrating multi-source stimulation has been proven to bring into play patients' subjective initiative to promote repair of damaged nerves after repeated clinical practice. In order to make patients actively participate in the rehabilitation training, the disclosure adopts a method combining functional electrical stimulation, thermal stimulation, and audio-visual fusion stimulation to bi-directionally repair a signal transmission chain of “nerve center-nerve pathway-muscle tissue” from working principles of the nerve system. A combination of external stimulation signals with the patients' rehabilitation status is also taken into account, which is an innovative rehabilitation treatment that differs from the most existing upper limb rehabilitation training devices.

### BRIEF DESCRIPTION OF DRAWINGS

FIG. 1 is a structural diagram of proactive assistance of linear drive according to the disclosure.

FIG. 2 is a schematic diagram of a rehabilitation system motor platform according to the disclosure.

FIG. 3 is a schematic diagram of a closed-loop myoelectric function rehabilitation system according to the disclosure.

FIG. 4 is a theory block diagram of a visual stimulation rehabilitation system according to the disclosure.

FIG. 5 is a theory block diagram of a nerve system according to the disclosure.

### DESCRIPTION OF REFERENCE NUMERALS

1-upper limb rehabilitation body; 2-rigid ring A; 3-rigid ring B; 4-rigid ring C; 5-rigid ring D; 6-rope; 7-micro servo motor; 8-closed-loop myoelectric function rehabilitation system; 9-visual stimulation rehabilitation system; 201-first rope knot; 301-second rope knot; 401-third rope knot; 501-fourth rope knot; 701-rehabilitation system motor platform; 702-pulley; 801-myoelectric signal collector; 802-electrical heating bandage; 803-stimulation electrode piece; 901-external visual stimulation module; 902-remodeling damaged nerve module; 903-cerebral cortex emitting command module; 904-stimulation muscle tissue module; 10-nerve system; 1001-nerve center module; 1002-nerve pathway module; 1003-muscle tissue module.

### DETAILED DESCRIPTION OF EMBODIMENTS

The technical solutions of the embodiments of the disclosure will be clearly and completely described below in combination with the attached drawings in the embodiments of the disclosure. Apparently, the described embodiments are only some of the embodiments of the disclosure and not all of the embodiments of the disclosure. Based on the embodiments of the disclosure, other embodiments obtained by those skilled in the art without creative work belong to the scope of protection of the disclosure.

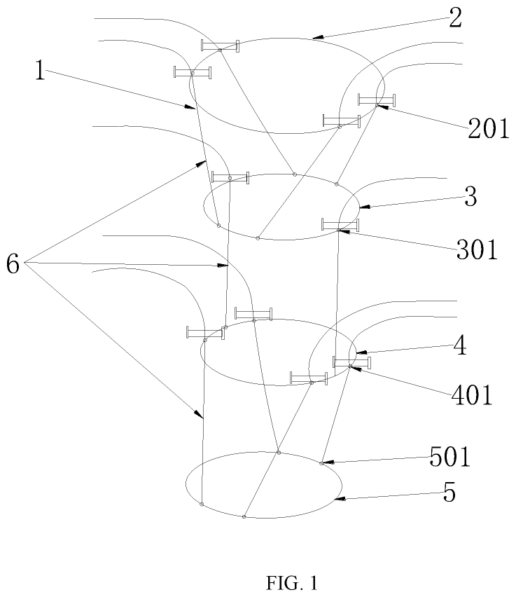

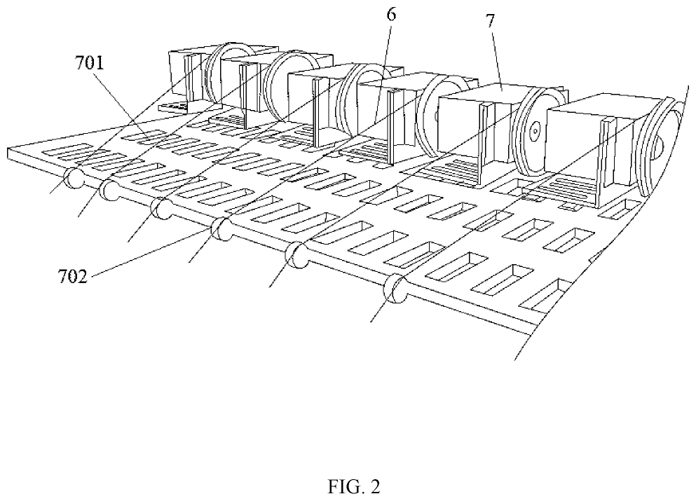

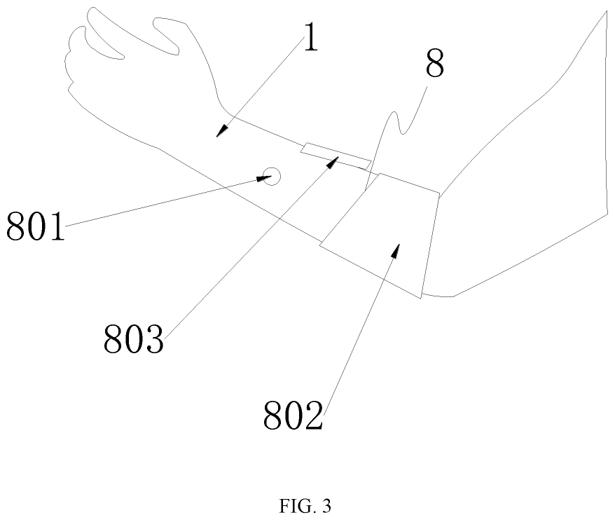

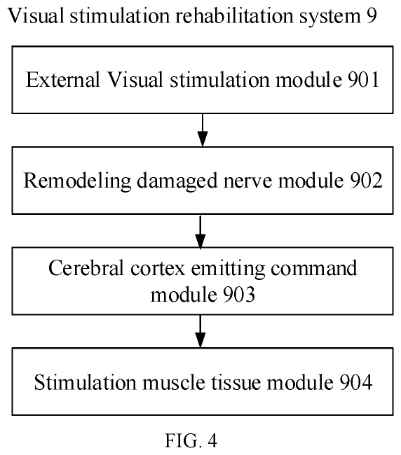

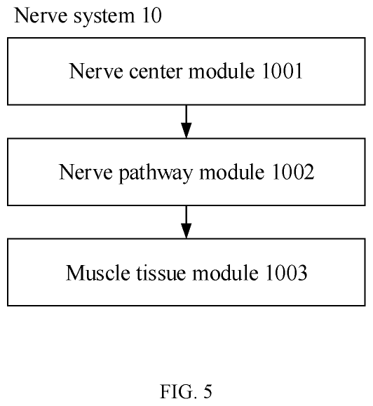

Reference from FIG. 1 to FIG. 5, an embodiment of the disclosure provides an upper limb rehabilitation training system integrating multi-source stimulation including an upper limb rehabilitation body 1, the upper limb rehabilitation body 1 is provided with a rigid ring A2, a rigid ring B3, a rigid ring C4 and a rigid ring D5. The rigid ring A2, the rigid ring B3, the rigid ring C4 and the rigid ring D5 are fixedly provided with first rope knots 201, second rope knots 301, third rope knots 401 and fourth rope knots 501 respectively. Four sets of ropes 6 are provided between the rigid ring A2 and the rigid ring B3, as well as the rigid ring C4 and the rigid ring D5. Three-degree-of-freedom movement of shoulder can be achieved by changing the length of the four sets of the ropes 6 between the rigid ring A2 and the rigid ring B3. Two sets of ropes 6 are provided between the rigid ring B3 and the rigid ring C4. Single-degree-of-freedom flexion and extension movement of elbow can be achieved by changing the length of the two sets of the ropes 6 between the rigid ring B3 and the rigid ring C4. Three-degree-of-freedom movement of wrist can be achieved by changing the length of the four sets of the ropes 6 between the rigid ring C4 and the rigid ring D5. Other ends of the ropes 6 are fixedly connected to the micro servo motors 7. The number of the ropes to assist driving the structure is 10, and ends of the ropes are connected to the rigid rings. When the micro servo motors 7 rotate, the length of the ropes 6 also changes, so as to realize proactive assistance. Patient puts an upper limb in the rigid ring A2, the rigid ring B3, the rigid ring C4 and the rigid ring D5 and changes the initial length of the ropes 6 to adapt. The upper limb rehabilitation body 1 is further provided with a closed-loop myoelectric functional rehabilitation system, a visual stimulation rehabilitation system 9 and a nerve system 10. The four sets of the ropes 6 are provided between the rigid ring A2 and the rigid ring B3, the two sets of the ropes 6 are provided between the rigid ring B3 and the rigid ring C4, and the four sets of the ropes 6 are provided between the rigid ring C4 and the rigid ring D5. The closed-loop myoelectric functional rehabilitation system 8 is provided with a myoelectric signal collector 801, an electric heating bandage 802 and a stimulation electrode piece 803. An output end of the myoelectric signal collector 801 is connected to an input end of the stimulation electrode piece 803, and an output end of the stimulation electrode piece 803 is connected to an input end of the electric heating bandage 802. The myoelectric signal collector 801 can collect an intensity of myoelectric signal in real time to illustrate a status of muscle. When the status of the muscle is significantly different from a normal value, the muscle tissue activates by stimulating the stimulation electrode piece 803 with additionally functional electrical stimulation to integrate collection, analysis and treatment of the myoelectric signal. The electric heating bandage 802 adopts hot compress temperature control technology, and is provided with a temperature sensor therein, which adopts hot compress temperature control technology (also referred to as the hot compress method with temperature controlled). The electric heating bandage 802 is wrapped around an upper limb, and after being energized, an electricity heating effect can generate heat, and the temperature sensor can realize a closed-loop control of temperature, so as to change temperature according to actual status to achieve efficient rehabilitation treatment. The visual stimulation rehabilitation system 9 is provided with an external visual stimulation module 901, a remodeling damaged nerve module 902, and a cerebral cortex emitting command module 903 and a stimulation muscle tissue module 904. An output end of the external visual stimulation module 901 is connected to an input end of the remodeling damaged nerve module 902. An output end of the remodeling damaged nerve module 902 is connected to an input end of the cerebral cortex emitting command module 903. An output end of the cerebral cortex emitting command module 903 is connected to an input end of the stimulation muscle tissue module 904. The nerve system 10 is provided with a nerve center module 1001, a nerve pathway module 1002 and a muscle tissue module 1003. The nerve center module 1001 is bi-directionally connected to an input end and an output end of the nerve pathway module 1002. The nerve pathway module 1002 is bi-directionally connected to an input end and an output end of the muscle tissue module 1003. And the output end of the muscle tissue module 1003 is connected to the input end of the stimulation muscle tissue module 904. The external visual stimulation nerve module 901 adopts one of technologies of VR and AR, the output end of the external visual module is connected to an output end of the nerve center module 1001. The micro servo motors 7 are provided on a rehabilitation system motor platform 701. A side of the rehabilitation system motor platform 701 is provided with pulleys 702, and the other ends of the ropes 6 are connected to the micro servo motors 7 through the pulleys 702.

At the same time, the content not described in detail in the disclosure belongs to the related art known to those skilled in the art.

When working, changing the length of the four sets of the ropes 6 provided between the rigid ring A2 and the rigid ring B3 can realize the three-degree-of-freedom movement of the shoulder, changing the length of the two sets of the ropes 6 provided between the rigid ring B3 and the rigid ring C4 can realize the single-degree-of-freedom flexion and extension movement of the elbow, and changing the length of the four sets of the ropes 6 provided between the rigid ring C4 and the rigid ring D5 can realize the three-degree-of-freedom movement of the wrist. When the micro servo motors 7 rotate, the length of the ropes 6 also changes, thus realizing proactive assistance. Patient puts an upper limb in the rigid ring A2, the rigid ring B3, the rigid ring C4 and the rigid ring D5 and changes the initial length of the ropes 6 to adapt. In the rehabilitation training, the myoelectric signal collector 801 can collect the intensity of the myoelectric signal in real time to illustrate the status of muscle. When the status of the muscle is significantly different from the normal value, the muscle tissue activates by stimulating the stimulation electrode piece 803 with additionally functional electrical stimulation to integrate the collection, the analysis and the treatment of the myoelectric signal. The electric heating bandage 802 is wrapped around the upper limb, after being energized, the electricity heating effect can generate heat, and the temperature sensor can realize the closed-loop control of temperature, so as to change the temperature according to the actual status to achieve the efficient rehabilitation treatment. The nerve system adopting human-computer interaction method to stimulate the patients brings into play the patients' subjective initiative, which is an important method to improve rehabilitation effect and shorten rehabilitation training period. The rehabilitation system adopts one of the technologies of VR and AR, combines the patients' ages, hobbies, life backgrounds and other features to build a more realistic personalized rehabilitation training scene, and combines rehabilitation tasks in the scene with the realistic position signal and the realistic intensity of the myoelectric signals to guarantee that the patients can fully devote themselves to participating in the rehabilitation training. The rehabilitation treatment requires a certain period, and muscle strength of the limbs, movement refinement will gradually improve with the rehabilitation process. Therefore, the proactive assistance larger, the external stimulation signal applied to the patients change accordingly, which follows a principle of “assistance on demand”. It is important to adjust according to the signal of the temperature sensor and patients' mental status. The disclosure dynamically adjusts the intensity of functional electrical stimulation and additional assistance based on the intensity of the myoelectric signal and the accuracy of limb movement to meet requirements of patients in different rehabilitation stages, and finally realize a progressive rehabilitation effect. The above description is working principles of the upper limb rehabilitation training system integrating multi-source stimulation.

It is noted that a description of “first”, “second”, etc., in the embodiments of the disclosure is used only to distinguish an entity or operation from another, and do not necessarily require or imply any actual relationship or order between those entities or operations. In addition, terms such as “include”, “comprise” or any other variation thereof are intended to cover a non-exclusive inclusion. Therefore, a process, method, thing, or device that includes a set of elements covers not only those elements, but also other elements not expressly listed, or that also covers a process, method, thing, or device that is inherent.

Although embodiments of the disclosure have been shown and described, it will be understood by those skilled in the art that a variety of variations, modifications, replacements and variants of these embodiments can be made without departing from principles and spirit of the disclosure. The scope of the disclosure is limited by attached claims and their equivalents.

What is claimed is:

1. An upper limb rehabilitation training system integrating multi-source stimulation, comprising:
an upper limb rehabilitation body;
first through fourth rigid rings, disposed on the upper limb rehabilitation body; wherein the first through fourth rigid rings are provided with first rope knots, second rope knots, third rope knots and fourth rope knots respectively, and connected to ends of ropes, and other ends of the ropes are connected to micro servo motors;
a closed-loop myoelectric functional rehabilitation system, disposed on the upper limb rehabilitation body;
a visual stimulation system, disposed on the upper limb rehabilitation body; and
a nerve system, disposed on the upper limb rehabilitation body.

2. The upper limb rehabilitation training system integrating multi-source stimulation according to claim 1, wherein four sets of the ropes are provided between the first rigid ring and the second rigid ring, two sets of the ropes are provided between the second rigid ring and the third rigid ring, and four sets of the ropes are provided between the third rigid ring and the fourth rigid ring.

3. The upper limb rehabilitation training system integrating multi-source stimulation according to claim 1, wherein the closed-loop myoelectric functional rehabilitation system comprises:
a myoelectric signal collector;
an electric heating bandage;
and a stimulation electrode piece, wherein an input end of the stimulation electrode piece is connected to an output end of the myoelectric signal collector, and an output end of the stimulation electrode piece is connected to an input end of the electric heating bandage.

4. The upper limb rehabilitation training system integrating multi-source stimulation according to claim 3, wherein the electric heating bandage adopts hot compress method with temperature controlled and is provided with a temperature sensor therein.

5. The upper limb rehabilitation training system integrating multi-source stimulation according to claim 1, wherein the visual stimulation system comprises:
an external visual stimulation module;
a remodeling damaged nerve module, wherein an input end of the remodeling damaged nerve module is connected to an output end of the external visual stimulation module;
a cerebral cortex emitting command module, wherein an input end of the cerebral cortex emitting command module is connected to an output end of the remodeling damaged nerve module; and
a stimulation muscle tissue module, wherein an input end of the stimulation muscle tissue module is connected to an output end of the cerebral cortex emitting command module.

6. The upper limb rehabilitation training system integrating multi-source stimulation according to claim 5, wherein the nerve system comprises:
a nerve center module;
a nerve pathway module, wherein an input end and an output end of the nerve pathway module are bi-directionally connected to the nerve center module; and
a muscle tissue module, wherein an input end and an output end of the muscle tissue module are bi-directionally connected to the nerve pathway module, and the output end of the muscle tissue module is connected to the input end of the stimulation muscle tissue module.

7. The upper limb rehabilitation training system integrating multi-source stimulation according to claim 6, wherein the external visual stimulation module adopts one of methods of VR (Virtual Reality) and AR (Augmented Reality), the output end of the external visual stimulation module is connected to an output end of the nerve center module.

8. The upper limb rehabilitation training system integrating multi-source stimulation according to claim 1, wherein the upper limb rehabilitation training system integrating multi-source stimulation, further comprises:
a rehabilitation system motor platform, installed with the micro servo motors; and
pulleys, disposed on a side of the rehabilitation system motor platform, wherein the other ends of the ropes are connected to the micro servo motors through the pulleys respectively.
